(19) United States
(12) Patent Application Publication
(10) Pub. No.: US 2009/0304607 A1
(43) Pub. Date: Dec. 10, 2009

(54) Antibacterial Flavor and Fragance Composition and Halitosis-Inhibition Flavor and Fragrance Composition and Oral Care Composition Containing the Same

(75) Inventors: Minoru HANADA, Hiratsuka-shi (JP); Fumihiro Yanaka, Hiratsuka-shi (JP); Yukihiro Kawakami, Hiratsuka-shi (JP)

Correspondence Address: SUGHRUE MION, PLLC, 2100 PENNSYLVANIA AVENUE, N.W., SUITE 800, WASHINGTON, DC, 20037, US

(73) Assignee: TAKASAGO INTERNATIONAL CORPORATION, Tokyo (JP)

(21) Appl. No.: 12/501,070

(22) Filed: Jul. 10, 2009

Related U.S. Application Data
(63) Continuation of application No. 10/514,129, filed on Nov. 12, 2004.

(30) Foreign Application Priority Data
Jun. 18, 2002 (JP) 2002-177134

(51) Int. Cl. A61K 8/18; A61Q 11/00

(52) U.S. Cl. 424/49

(57) ABSTRACT

A flavor and fragrance composition possessing an antibacterial activity effective against periodontal disease-causing bacteria and/or a halitosis-inhibition action capable of controlling production of volatile sulfides, which is safe for the human body, and containing at least one or more substances selected from food fragrance materials such as hexylaldehyde, caryophyllene alcohol, cinnamicaldehyde, dihydroeugenol, farnesol, dihydrofarnesol, hinokitiol, isoeugenol, γ-undecalactone, d-limonene, o-methoxycinnamicaldehyde, β-pinene, γ-terpinene, terpinolene, orange oil, nutmeg oil, and grapefruit oil, and an oral care composition containing the same.

This is a continuation of application Ser. No. 10/514,129 filed Nov. 12, 2004, which is a 371 National Stage Application of PCT Application No. PCT/JP03/07745 filed Jun. 18, 2003, which claims foreign priority to Japanese Patent Application No. 2002-177134 filed on Jun. 18, 2002. The entire disclosures of the prior applications are hereby incorporated by reference.

### TECHNICAL FIELD

The present invention relates to an antibacterial flavor and fragrance composition which is excellent in safety and exhibits an inhibitory action effectively on periodontal disease-causing bacteria. Moreover, it relates to a halitosis-inhibition flavor and fragrance composition inhibiting production of volatile sulfides which are bad-smelling substances in halitosis. Furthermore, the invention relates to an oral care composition containing the above antibacterial flavor and fragrance composition or halitosis-inhibition flavor and fragrance composition.

### BACKGROUND ART

In the oral cavity, several hundred kinds of aerobic and anaerobic microorganisms such as bacteria and fungi inhabit and closely take part in diseases in the oral cavity. The growth of these pathogenic microorganisms is induced by some causes, and induces various diseases such as dental caries (decayed tooth), periodontal disease, stomatitis, and halitosis. In particular, dental caries and periodontal disease are two main diseases in the oral cavity and a serious concern has been focused on the prevention and treatment of these diseases. As periodontal disease-causing bacteria, Fusobacterium nucleatum, Porphyromonas gingivalis, Prevotella intermedia, and the like are known.

On the other hand, it has been widely known that essential oils and fragrance materials as the ingredients possess antibacterial and antifungal activities (Tohru Asagoe, Nihon Keshohin Gijutsusha Kaishi, Vol. 34, pp. 25-46, 2000). Specifically, it has been reported that hinokitiol, cinnamicaldehyde, thymol, eugenol, and the like possess an antibacterial activity against the above bacteria derived from the oral cavity (Bull. Tokyo Dent. Coll., 30(3), 129-135 1989). Moreover, spice extracts such as allspice have been reported to possess an antifungal activity against Fusobacterium nucleatum (JP-B-62-58327). Furthermore, there is a report (Japanese Patent No. 3154285) that a Mentha Herb extract possesses inhibitory effecton collagenase and thus an effect of preventing and treating periodontal disease is expectable.

However, most of these antibacterial activities are determined in accordance with the minimum inhibitory concentration-measuring method using agar media or liquid media. In the minimum inhibitory concentration-measuring method, since a test bacterium and a sample are present together in a medium during the experimental period, the resulting antibacterial activity exhibits no difference whether the activity is bactericidal one or bacteriostatic one.

In the case that a fragrance material is actually used as a flavor, however, the period during which the fragrance material is in contact with target bacteria is limited depending on products. For example, in the case of toothpaste, the contact period of which is only that of brushing, diluted with saliva and rinsed out with water. The situation is similar in the case of mouthwash, and in the case of chewing gum, when chewing of the gum is over or all the flavor is completely eluted from the gum into saliva, the contact with the target bacteria occurs no more. Therefore, a substance requiring a long period of time for achieving sterilization cannot exhibit a desired effect even if it possesses a bacteriostatic or bactericidal activity. When such a situation is considered, the results of the minimum inhibitory concentration-measuring method do not predict an activity when actually used.

Therefore, in order to obtain one exhibiting the effect by mixing it with an oral care composition, an antibacterial substance should exhibit the activity during the contact with target bacteria over only a certain period of time. A similar test has been carried out on Listerine which is a commercially available mouthwash and its strong activity has been reported (Sikai Tenbo, 76, 1459-1466, 1990). In addition, there is a report (WO99/32075) that the fragrance material used in Listerine is applied to tooth paste.

On the other hand, there is a patent (WO01/24769) that a fragrance composition exhibits activity against aerobic bacteria such as Staphylococcus aureus and Escherichia coli during the contact over such a short period. Moreover, with increase in inclination for cleanliness in recent years, people who worry about halitosis, body odor, and the like have increased. Halitosis includes physiological halitosis and halitosis caused by meals or nonessential grocery items even observed in healthy individuals and also halitosis derived from pathological causes, the latter being further classified halitosis derived from diseases in the oral cavity and halitosis derived from systemic diseases.

Among these kinds of halitosis, the most unpleasant feeling is caused by the halitosis derived from diseases in the oral cavity. It is considered that the halitosis derived from diseases in the oral cavity is caused by volatile sulfides (hydrogen sulfide, methyl mercaptan, dimethyl sulfide, etc.) produced by the metabolism of dental bacteria, especially anaerobic bacteria using food residues, mucosal exfoliates, and exudates from the site of inflammation as protein sources and also bad-smelling substances such as indole and skatole produced by similar metabolism (Nihon Shikaishikai Zasshi 29(3), 228-235, 1976). Of these, it is known that the concentration of methyl mercaptan particularly correlates with the strength of halitosis.

Therefore, in order to reduce or eliminate halitosis, the following methods may be considered: (1) removal of bad-smelling ingredients such as methyl mercaptan produced, (2) masking with the other fragrance material, (3) removal of smear in the oral cavity (nutrients for dental bacteria), (4) inhibition of the growth of halitosis-causing bacteria or sterilization thereof, (5) inhibition of the production of methyl mercaptan by halitosis-causing bacteria, and the like. However, the methods (1), (2), and (3) cannot be said as fundamental methods, and it is needless to say that the method (4) or (5) is superior to the other methods.

In order to control the halitosis, it has been carried out to add copper chlorophyllin sodium having a deodorant effect to an oral care composition. Moreover, addition of flavones (Shokuhin Kogyo, 38(4), 70-78, 1992), tea catechins (Shokuhin Kogyo, 38(18), 28-33, 1992), rosaceous plant extracts (Nihon Nogei Kagakukaishi, 66(10), 1475-1479, 1992), extracts of Atractylodes japonica, Aesculus turbinata, and Thujopsis dolabrata, (Japanese patent No. 2950674), plant extracts (J. Odor Research and Eng., 31(2), 91, 2000), or the like has been reported. However, the effects of these active ingredients are limited and a relatively large amount thereof is required, so that these methods are hardly said as fundamental methods.

It is known that anaerobic culture of saliva in a tightly sealed vessel produces volatile sulfides such as methyl mercaptan (Arch. Oral Biol., 9, 47-53, 1964). Moreover, it is also known that volatile sulfides produced by culturing saliva as it is or a discharged liquid after mouth-washing mixed with methionine as a sulfur-containing amino acid is inhibited by tea catechins (Nihon Shokuhin Kogyo Gakkaishi, 38(12), 1098-1102, 1991).

On the other hand, the volatile sulfides production is inhibited by plant extracts (Shuuki no kennkyuu, 31 (2), 91-96, 2000; JP-A-2002-114660), and various fragrance materials (JP-A-2001-348308) when inoculated washed cells of Fusobacterium nucleatum or Porphyromonas gingivalis, known to produce volatile sulfides, to a medium or buffer containing methionine or cysteine.

Moreover, for the purpose of removing bacteria in the oral cavity, an antibiotic or synthetic antibacterial agent is administered, but problems that the treatment results in appearance of resistant bacteria, toxicity derived from a long-term use, and imbalance of enteric bacteria owing to the strong activity have been pointed out. Therefore, it is desired to search a substance less toxic and excellent in an antibacterial activity or a substance effectively inhibiting halitosis, especially the production of volatile sulfides including methyl mercaptan.

### DISCLOSURE OF THE INVENTION

Accordingly, an object of the invention is to provide an antibacterial flavor and fragrance composition and/or a halitosis-inhibition flavor and fragrance composition possessing an antibacterial activity and/or a halitosis-inhibition activity which satisfy the above requirements, and an oral care composition containing the same.

As a result of extensive studies for solving the above problems, the present inventors have found that among food fragrance ingredients and natural fragrance materials the safety of which is confirmed during a long-term use experience, we find out those effective substances having excellent antimicrobial activities or those effectively inhibiting halitosis, especially inhibiting the production of volatile sulfides including methyl mercaptan. Furthermore, they have found that among fragrance ingredients and natural fragrance materials frequently used in foods, there exist substances excellent in an antibacterial activity even during the contact with target bacteria over a short period of time or substances effectively inhibiting halitosis, especially the production of volatile sulfides including methyl mercaptan. Based on these findings, the invention has been accomplished.

Namely, the invention includes the following each invention.

(1) An antibacterial flavor and fragrance composition for adding to an oral care composition comprising at least one or more substances selected from hexylaldehyde, caryophyllene alcohol, cinnamicaldehyde, dihydroeugenol, farnesol, dihydrofarnesol, hinokitiol, isoeugenol, γ-undecalactone, d-limonene, o-methoxycinnamicaldehyde, β-pinene, γ-terpinene, terpinolene, orange oil, nutmeg oil, grapefruit oil, dill weed oil, pine needle oil, spearmint oil, tangerine oil, sweety oil, lemon oil, lime oil, Mentha Herb oil, and mandarin oil.
(2) A halitosis-inhibition flavor and fragrance composition for adding to an oral care composition comprising at least one or more substances selected from hexylaldehyde, allyl cyclohexylpropionate, allyl enanthate, α-amylcinnamicaldehyde, caryophyllene alcohol, l-carvone, cinnamicaldehyde, cinnamicaldehyde dimethyl acetal, citral, menthoxypropanediol, dihydroeugenol, ethyl salicylate, methyl salicylate, eugenol, farnesol, dihydrofarnesol, hinokitiol, isoeugenol, γ-decalactone, γ-undecalactone, d-limonene, linalyl acetate, menthol, o-methoxycinnamicaldehyde, methyl-isoeugenol, β-pinene, γ-terpinene, terpinolene, thymol, orange oil, nutmeg oil, grapefruit oil, pine needle oil, spearmint oil, tangerine oil, sweety oil, peppermint oil, lemon oil, eucalyptus oil, lime oil, Mentha Herb oil, and mandarin oil.
(3) An oral care composition comprising the antibacterial flavor and fragrance composition described in the above (1).
(4) An oral care composition comprising the halitosis-inhibition flavor and fragrance composition described in the above (2).

### BEST MODE FOR CARRYING OUT THE INVENTION

The present invention will be described in more detail.

The antibacterial flavor and fragrance composition for adding to an oral care composition according to the invention contains at least one or more specific fragrance materials selected from hexylaldehyde, caryophyllene alcohol, cinnamicaldehyde, dihydroeugenol, farnesol, dihydrofarnesol, hinokitiol, isoeugenol, γ-undecalactone, d-limonene, o-methoxycinnamicaldehyde, β-pinene, γ-terpinene, terpinolene, orange oil, nutmeg oil, grapefruit oil, dill weed oil, pine needle oil, spearmint oil, tangerine oil, sweety oil, lemon oil, lime oil, Mentha Herb oil, and mandarin oil.

The spearmint oil includes spearmint oil native, spearmint oil scotch, and the like, lime oil includes lime oil, distilled lime oil, and the like, and mandarin oil includes mandarin red oil and the like.

Moreover, the halitosis-inhibition flavor and fragrance composition for adding to an oral care composition according to the invention contains at least one or more specific fragrance materials selected from hexylaldehyde, allyl cyclohexylpropionate, allyl enanthate, α-amylcinnamicaldehyde, caryophyllene alcohol, l-carvone, cinnamicaldehyde, cinnamicaldehyde dimethyl acetal, citral, menthoxypropanediol, dihydroeugenol, ethyl salicylate, methyl salicylate, eugenol, farnesol, dihydrofarnesol, hinokitiol, isoeugenol, γ-decalactone, γ-undecalactone, d-limonene, linalyl acetate, menthol, o-methoxycinnamicaldehyde, methyl-isoeugenol, β-pinene, γ-terpinene, terpinolene, thymol, orange oil, nutmeg oil, grapefruit oil, pine needle oil, spearmint oil, tangerine oil, sweety oil, peppermint oil, lemon oil, eucalyptus oil, lime oil, Mentha Herb oil, and mandarin oil.

In these specific fragrance materials, hexylaldehyde, caryophyllene alcohol, cinnamicaldehyde, dihydroeugenol, farnesol, dihydrofarnesol, hinokitiol, isoeugenol, γ-undecalactone, d-limonene, o-methoxycinnamicaldehyde, β-pinene, γ-terpinene, terpinolene, orange oil, nutmeg oil, grapefruit oil, pine needle oil, spearmint oil, tangerine oil, sweety oil, lemon oil, lime oil, Mentha Herb oil, and mandarin oil are particularly preferable specific fragrance materials because they possess both of antibacterial effect and halitosis-inhibition effect.

Flavor and fragrance compositions commonly used as food fragrance ingredients can be further added to the antimicrobial and bad breath-controlling composition and used as the oral composition. As the other fragrance materials used for the food additives such as synthetic fragrance materials usable for the addition, a common food fragrance ingredient such as various synthetic fragrance materials, natural essential oils, synthetic essential oils, citrus oils, animal fragrance materials, and the like are mentioned. Of these materials, it is preferred to use a wide range of fragrance ingredients described in “Shokuhin Kouryou handbook, edited by Nihon Kouryou Kougyoukai, published by Shokuhin kagaku Shinbunsha on Feb. 15, 1990”, for oral compositions and foods.

Examples of the oral care composition of the invention include toothpaste; mouthwash or oral detergents; foods such as chewing gum, candies, troches, gummi jelly, and tablets; and the like.

The amount of the antibacterial flavor and fragrance composition and/or halitosis-inhibition flavor and fragrance composition of the invention to be mixed with the oral care composition is not particularly limited but desirably, the amount is usually from 0.005 to 10% (% by weight, the same shall apply hereinafter), particularly from 0.02 to 3% based on the total oral care composition. When the amount is less than 0.005%, the antibacterial effect is sometimes not sufficiently exhibited, and when the amount is more than 10%, flavor of the oral care composition is sometimes deteriorated.

In addition to the aforementioned antibacterial flavor and fragrance composition or halitosis-inhibition flavor and fragrance composition, the oral care composition of the invention can be mixed with suitable ingredient(s) depending on the purpose and kind of the composition.

For example, toothpaste can be mixed with an abrasive such as calcium secondary phosphate dihydrate, calcium carbonate, or silicic acid anhydride; a surfactant such as sodium lauryl sulfate or a polyoxyethylenealkyl sulfate salt; a foaming agent such as a sucrose fatty acid ester, a maltose fatty acid ester, or sodium lauroylsarcosine; a wetting agent such as glycerin, sorbit, or propylene glycol; a caking agent such as carboxymethyl cellulose, sodium alginate, carrageenan, or xanthan gum; and the like.

Furthermore, toothpaste can be mixed with tranexamic acid, dipotassium glycyrrhizinate, dl-α-tocopherol acetate, or the like as an anti-inflammatory agent; aluminum lactate or the like as an astringent; saccharin sodium, stevioside, glycyrrhizin, xylitol, or the like as a sweetener; a natural fragrance raw material such as angelica root oil, basil oil, bay oil, bergamot oil, bitter almond oil, calamus oil, camomile oil, caraway oil, cardamon oil, Japanese quassia oil, cinnamon oil, clary sage oil, clove oil, cognac oil, coriander oil, estragon oil, funnel oil, geranium oil, guaiac wood oil, juniper oil, laurel leaf oil, lavender oil, mace oil, majoran oil, myrrh oil, neroli oil, palmarosa oil, petigrain oil, pimento oil, rose oil, rosemary oil, sandal wood oil, staranise oil, sage oil, thyme oil, wintergreen oil, or ylang-ylang oil as a flabor; and a fragrance material such as anethole, anisaldehyde, benzaldehyde, ethyl benzoate, methyl benzoate, benzyl benzoate, borneol, bornyl acetate, carvacrol, citronellol, ethyl methylphenylglycidate, ethylvanillin, cineole, geraniol, heliotropin, ionone, irone, linalool, menthyl acetate, methyl amyl ketone, methyl cinnamate, nerol, nonyl acetate, phenyl methyl ether, rhodinol, phenyl ethyl alcohol, terpineol, vanillin, maltol, or ethylmaltol, and also can be mixed with a benzoic acid derivative such as sodium benzoate, sodium p-oxybenzoate, or butyl p-oxybenzoate, and sodium salicylate, and the like as a preservative.

In this connection, in addition to the antibacterial flavor and fragrance composition or the halitosis-inhibition flavor and fragrance composition, as active ingredients, a cationic bactericide such as chlorhexidine or benzalconium chloride; a phenolic compound such as triclosan; an enzyme such as dextranase, lysozyme, lytic enzyme, or superoxide dismutase; a fluoride such as sodium fluoride; and the like can be mixed.

Mouthwash and oral detergents can be prepared by mixing the composition together with a surfactant, a flavor, and a sweetener similar to those used in toothpaste.

As foods, the composition can be mixed with chewing gum, candies, troches, gummi jelly, and tablets; and the like which can be generally stay in the mouth for a certain period of time in an amount of 0.005 to 5%, more preferably 0.02 to 2%.

### EXAMPLES

The invention will be described in further detail with reference to the following Examples, but the present invention is not limited in any way to these examples.

### Example 1

### Measurement of Bactericidal Activity

The measurement of bactericidal activity of flavor and fragrance composition of the invention was carried out in accordance with the method of Quantitative Suspension Test (British Standard EN 1276, 1997).

The strain used in the bactericidal activity was obtained from The Institute of Physical and Chemical Research which was known to be a pathogen of periodontal disease and also known to produce volatile sulfides.

The following shows an abbreviated code (hereinafter described using the abbreviated code in this text) and name of the strain.

Test Strain

| Abbreviated Code | Name of Strain |
|---|---|
| Fn-1 | Fusobacterium nucleatum JCM 6328 |

Fn-1 was cultured on slants of GAM medium (Nissui Pharmaceutical Co. Ltd.) and added physical saline to make a cell suspension. In detail, to each slant cultured at 37° C. for 24 hours under an anaerobic condition using BBL gas-pack anaerobic system, 2 ml of physiological saline was added and scraped off the microbes with a transfer loop to make a cell suspension. This operation was repeated twice, and the combined suspension was centrifugalized, added again a half amount of physiological saline to the precipitate to make the suspension. The suspension was filtered through two sheets of sterile gauze to remove agar pieces and fibrous cells. The filtrate was centrifugalized again, the precipitate was re-suspended in physiological saline to use for the tests. From five slants, nearly 5 ml of homogeneous cell suspension of 1×10⁷ to 10⁸ CFU (colony Forming Unit)/ml was obtained.

As for test solutions, fragrance samples were dissolved in equal weight of dimethylformamide (DMF) and were diluted with ethanol to necessary concentrations (volume/volume).

Each test solution (30 μl) was added to sterile distilled water and stirred or subjected to ultrasonic treatment to dissolve sample or to form a homogeneous suspension.

To each solution or homogeneous suspension, 333 μl of previous cell suspension of Fn-1 was added and stirred. After 30 seconds and 1 minute, each 333 μl was added to 3 ml of D/E Neutralization broth (DIFCO) (10 fold dilution), 100 fold and 1000 fold dilution liquids were made similarly. Each 50 μl of the liquid was spread on 15 ml of Trypticase soy agar plates using Autoplate 4000 (Spiral Biotec Co. Ltd., MD, USA) and were cultivated at 37° C. for 40 hours under anaerobic condition. After the cultivation, colonies were counted by Q-Count (Spiral Biotec Co. Ltd., MD, USA) to determine CFU/ml. This method is referred to as a CFU measuring method in accordance with Quantitative Suspension Test (British Standard EN 1276, 1997).

Suspension, as a control, consisted of 3 ml of water and 333 μl of the cell suspension, and also suspension as a reference, consisted of 3 ml of water, 30 μl of ethanol and 333 μl of the cell suspension was employed to determine CFU/ml thereof.

The judgments of the results were represented by Log unit of the decrease of the number of the bacteria after the contact with fragrance materials after 30 seconds and 1 minutes comparing with the control. When no colony was observed at each dilution step, results were shown by attaching > mark to the Log value obtained in the control, because the numbers of the colony were thought to be 100 or less. The value 1.00 was shown that CFU was reduced to 1/10, and the value 3.00 was shown that CFU was reduced to 1/1000.

The results of this test were shown in Table 1. “30 sec” and “1 min” were shown the sampling time, and “Log inc.” was shown the mean Log value of the control. The reason why this value was described was that the values of the control were different in each experiment.

TABLE 1

| | | Concen- | Results of | | |
|---|---|---|---|---|---|
| | | tration | Judgment | | Log |
| No. | Sample | ppm | 30 sec | 1 min | Inc. |
| 1 | Hexylaldehyde | 300 | 0.92 | 1.21 | 6.45 |
| 2 | Caryophyllene alcohol | 500 | 1.13 | 2.25 | 6.18 |
| 3 | Cinnamicaldehyde | 2000 | >3.42 | >3.42 | 5.42 |
| 4 | Dihydroeugenol | 2000 | >3.42 | >3.42 | 5.42 |
| 5 | Farnesol | 100 | 3.70 | >4.18 | 6.18 |
| 6 | Dihydrofarnesol | 300 | >3.42 | >3.42 | 5.42 |
| 7 | Hinokitiol | 1000 | >5.24 | >5.24 | 7.24 |
| 8 | Isoeugenol | 500 | >3.93 | >3.93 | 5.93 |
| 9 | γ-Undecalactone | 50 | >5.15 | >5.15 | 7.15 |
| 10 | d-Limonene | 500 | 0.93 | 1.50 | 7.15 |
| 11 | o-Methoxycinnamicaldehyde | 1000 | 2.61 | 3.85 | 6.45 |
| 12 | β-Pinene | 500 | 0.71 | 1.12 | 6.20 |
| 13 | γ-Terpinene | 500 | 0.94 | 1.26 | 6.20 |
| 14 | Terpinolene | 100 | 1.16 | 1.63 | 6.20 |
| 15 | Orange oil | 2000 | 1.20 | 2.02 | 6.16 |
| 16 | Nutmeg oil | 300 | 1.69 | 2.24 | 6.18 |
| 17 | Grapefruit oil | 2000 | 2.86 | 3.69 | 6.16 |
| 18 | Dill weed oil | 200 | 0.67 | 1.11 | 6.18 |
| 19 | Pine needle oil | 500 | 0.51 | 1.00 | 6.18 |
| 20 | Spearmint oil native | 2000 | >4.16 | >4.16 | 6.16 |
| 21 | Spearmint oil scotch | 2000 | 1.92 | 3.86 | 6.16 |
| 22 | Tangerine oil | 2000 | 1.73 | 2.32 | 6.18 |
| 23 | Sweety oil | 2000 | 1.24 | 2.00 | 6.16 |
| 24 | Lemon oil | 2000 | 1.15 | 1.85 | 6.16 |
| 25 | Lime oil | 2000 | 0.70 | 1.16 | 6.20 |
| 26 | Distilled lime oil | 2000 | 1.70 | 2.11 | 6.20 |
| 27 | Mentha Herb oil | 2000 | 1.37 | 3.09 | 6.16 |
| 28 | Mandarin red oil | 500 | 1.02 | 1.56 | 6.18 |

As shown in Table 1, these fragrance materials are able to reduce the test bacterium to 1/10 in number by contact with that for 30 seconds or 1 minute, and it is shown that they were exhibited sufficient bactericidal activities.

### Example 2

### Inhibition of Volatile Sulfide Production from Saliva

A test of inhibitory activity of the fragrance materials on volatile sulfide production from saliva was carried out according to the following method. Saliva was collected in a 50 ml of sterile centrifuge tube from testee without brushing teeth in the morning between 9 to 10 o'clock, and was cooled on ice until use. Each of 2 ml of the saliva was put into sterile test tube with screw cap aseptically. Saliva only was used as control and that mixed with 20 μl of ethanol was used as reference. Fragrance samples were prepared as in the case of Example 1 and 20 μl of each sample was added in saliva.

After the addition of samples, the inner air was replaced nitrogen in a stream of nitrogen gas at 0.5 atm under stirring with a voltex mixer. Cultivation was carried out at 37° C. for 3 hours and cooled on ice until use. Volatile sulfides were analyzed by a gas chromatograph (FPD-GC) with a flame photometer. In this connection, the samples after the culture were stored in ice-water until their analysis.

### (Measuring Conditions of Gas Chromatography)

Column: 6 m×4 mm i.d. (glass packed column)
Column liquid phase: 20% DNP on 80/100 mesh Chromosorb W AW DMCS
Column temperature: 100° C.
Carrier gas (flow rate): N₂ (40 ml/min)
Injection temperature: 120° C.
Detector: FPD (flame photometric detector; fitted with 393 nm filter for sulfur compound detection)
Detector temperature: 150° C.

When 1 ml of head space after the saliva culture was analyzed under the above conditions, unidentified volatile sulfides were detected in addition to hydrogen sulfide, methyl mercaptan, and dimethyl sulfide. However, since the sulfides other than methyl mercaptan vary depending on the day which saliva was collected and sometimes were not produced, the production inhibition rate (hereinafter, inhibition rate) of methyl mercaptan alone is shown as a result in Table 2.

The inhibition rate of methyl mercaptan was determined according to the following equation:

Inhibition rate (%) of methyl mercaptan={(Blank−Sample)/Blank}×100

Blank and Sample in the equation represent the quantities of producing methyl mercaptan in respective cases.

TABLE 2

| | | Concentration | CH₃SH |
|---|---|---|---|
| No. | Name of Sample | ppm | Inhib. % |
| 1 | Hexylaldehyde | 300 | 82% |
| 2 | Allyl cyclohexylpropionate | 500 | 92% |
| 3 | Allyl enanthate | 1000 | 100% |
| 4 | α-Amylcinnamicaldehyde | 500 | 99% |
| 5 | Caryophyllene alcohol | 500 | 100% |
| 6 | L-Carvone | 1000 | 100% |
| 7 | Cinnamicaldehyde | 2000 | 100% |
| 8 | Cinnamicaldehyde dimethyl acetal | 1000 | 100% |
| 9 | Citral | 500 | 100% |
| 10 | Menthoxypropanediol | 1000 | 100% |
| 11 | Dihydroeugenol | 2000 | 100% |
| 12 | Ethyl salicylate | 1000 | 100% |
| 13 | Eugenol | 500 | 100% |
| 14 | Farnesol | 100 | 100% |
| 15 | Dihydrofarnesol | 300 | 100% |
| 16 | Hinokitiol | 1000 | 100% |
| 17 | Isoeugenol | 500 | 100% |
| 18 | γ-Decalactone | 100 | 59% |
| 19 | γ-Undecalactone | 100 | 70% |
| 20 | d-Limonene | 3000 | 100% |
| 21 | Linalyl acetate | 500 | 43% |
| 22 | Menthol | 3000 | 100% |
| 23 | O-Methoxycinnamicaldehyde | 1000 | 100% |
| 24 | Methyl-isoeugenol | 2000 | 100% |
| 25 | Methyl salicylate | 1000 | 100% |
| 26 | β-Pinene | 500 | 59% |
| 27 | γ-Terpinene | 500 | 66% |
| 28 | Terpinolene | 100 | 36% |
| 29 | Thymol | 300 | 100% |
| 30 | Orange oil | 2000 | 100% |
| 31 | Nutmeg oil | 300 | 100% |
| 32 | Grapefruit oil | 500 | 100% |
| 33 | Pine needle oil | 500 | 97% |
| 34 | Spearmint oil native | 2000 | 100% |
| 35 | Spearmint oil scotch | 2000 | 100% |
| 36 | Tangerine oil | 3000 | 100% |
| 37 | Sweety oil | 2000 | 100% |
| 38 | Peppermint oil | 2000 | 100% |
| 39 | Lemon oil | 2000 | 100% |
| 40 | Eucalyptus oil | 2000 | 100% |
| 41 | Lime oil | 2000 | 100% |
| 42 | Distilled lime oil | 2000 | 100% |
| 43 | Mentha Herb oil | 2000 | 100% |
| 44 | Mandarin red oil | 2000 | 96% |

As shown in Table 2, it was understood that many fragrance materials inhibit the production of methyl mercaptan.

### Example 3

From the results obtained in Examples 1 and 2, two types of spearmint type and peppermint type of halitosis-inhibition blended fragrance material were prepared according to the following formulation. The formulation of the fragrance materials are shown in Table 3.

TABLE 3

| | Spearmint | Peppermint | Peppermint |
|---|---|---|---|
| | type | type A | type B |
| Spearmint oil native | 28 | — | 10 |
| l-Calvon | 14 | — | — |
| Thymol | 3 | 3 | 3 |
| Dihydrofarnesol | 3 | 3 | 3 |
| Grapefruit oil | 10 | 10 | 5 |
| Orange oil | 10 | 10 | 5 |
| γ-Undecalactone | 0.5 | 0.5 | 0.5 |
| Dihydroeugenol | 3 | 3 | 3 |
| Other flavors | 28.5 | — | — |
| Peppermint oil | — | 44.5 | 44.5 |
| Flavor base (Takasago) | — | 26 | 26 |
| Total | 100 | 100 | 100 |

The antibacterial activity of three types of the blended fragrance material prepared and 44.5 parts by weight of peppermint oil and 26 parts by weight of a flavor base (mfd. by Takasago International Corporation) used therein against Fn-1 were confirmed according to the method of Example 1. The results are shown in Table 4.

TABLE 4

| | | Results of | | |
|---|---|---|---|---|
| | | Judgment | | Log |
| | Concentration % | 30 sec | 1 min | Inc. |
| Spearmint type | 0.2 | >5.06 | >5.06 | 7.06 |
| Peppermint type A | 0.2 | >5.06 | >5.06 | 7.06 |
| Peppermint type B | 0.2 | >5.06 | >5.06 | 7.06 |
| Spearmint flavor base | 0.2 | 0.14 | −0.11 | 7.06 |

As shown in Table 4, the three types of the prepared mixed fragrance materials exhibited a strong antibacterial activity against Fn-1 at a concentration of 0.2%, but the spearmint flavor base exhibited no antibacterial activity at the same concentration.

Then, a test of inhibitory activity of the three kinds of the prepared mixed fragrance materials on the production of volatile sulfides by saliva was carried out according to the method of Example 2. At the same time, change of the number (CFU/ml) of anaerobic bacteria in saliva after 3 hours of culture was also measured.

Collected saliva was cultured in the same manner as in Example 2, and volatile sulfides produced were analyzed. After completion of the analysis, 333 μl of the cultured saliva was sampled and diluted with 3 ml of physiological saline (10 times), followed by similar dilution until ten thousand times. Then, the number (CFU/ml) of viable anaerobic bacteria in the saliva was measured in the same manner as in Example 1. During the culture, the original saliva was cooled on ice and the number of the anaerobic bacteria was measured in a similar manner to the case of the cultured saliva.

The results are shown in Tables 5 and 6. With regard to the viable anaerobic bacteria, the increase or decrease from the number (47 million) of bacteria in the original saliva was represented by percentage (Table 5). Moreover, with regard to the volatile sulfides, the concentration (ppb) of produced hydrogen sulfide and methyl mercaptan was investigated and inhibition rate in comparison with the concentration after 3 hours of the cultivation of the saliva was represented by percentage (Table 6). In this connection, the designation of − (minus) means an increase in comparison with the blank.

TABLE 5

| | Concen- | CFU % | H₂S | | CH₃SH | |
|---|---|---|---|---|---|---|
| | tration | decrease | | Inhibi- | | Inhibi- |
| Sample | ppm | rate | ppb | tion % | ppb | tion % |
| Saliva | — | 4.70E+07 | Not analyzed | | | |
| Saliva after 3 | — | −22.6 | 1500 | — | 590 | — |
| hours of culture | | | | | | |
| Saliva + 1% | — | −23.4 | 1500 | 0 | 380 | 36 |
| ethanol | | | | | | |
| Spearmint type | 1000 | 89.8 | 0 | 100 | 0 | 100 |
| Spearmint type | 250 | 12.4 | 230 | 85 | 170 | 71 |
| Spearmint type | 62.5 | 5.4 | 1400 | 7 | 440 | 25 |

TABLE 6

| | Concen- | CFU % | H₂S | | CH₃SH | |
|---|---|---|---|---|---|---|
| | tration | decrease | | Inhibi- | | Inhibi- |
| Sample | ppm | rate | ppb | tion % | ppb | tion % |
| Saliva | — | 7.06E+07 | Not analyzed | | | |
| Saliva after 3 | — | −46.2 | 1500 | — | 360 | — |
| hours of culture | | | | | | |
| Saliva + 1% | — | −64.7 | 1400 | 7 | 240 | 33 |
| ethanol | | | | | | |
| Peppermint type A | 1000 | 90.6 | 0 | 100 | 0 | 100 |
| Peppermint type A | 250 | 29.9 | 200 | 87 | 85 | 76 |
| Peppermint type B | 1000 | 94.0 | 0 | 100 | 10 | 97 |
| Peppermint type B | 250 | 30.5 | 800 | 47 | 280 | 22 |

As shown in Tables 5 and 6, it was understood that the mixed fragrance materials of spearmint type and peppermint type reduced the number (CFU/ml) of the anaerobic bacteria in the saliva to about 1/10 at a concentration of 1000 ppm, and also nearly completely inhibited the production of volatile sulfides.

### Example 4

A toothpaste with the following formulation was produced in a usual manner.

| Mixing components | % by weight |
|---|---|
| Calcium phosphate | 30 |
| Glycerin | 10 |
| Sorbitol | 20 |
| Sodium lauryl sulfate | 1.5 |
| Sodium carboxymethyl cellulose | 1.0 |
| Carrageenan | 0.1 |
| Saccharin sodium | 0.1 |
| Spearmint type mixed fragrance | 1.0 |
| material (Example 3) | |
| Sodium benzoate | 0.3 |
| Water | balance |

### Example 5

A mouthwash with the following formulation was produced in a usual manner.

| Mixing components | % by weight |
|---|---|
| Ethanol | 10.0 |
| Glycerin | 5.0 |
| Citric acid | 0.01 |
| Sodium citrate | 0.1 |
| Polyoxyethylene hardened caster oil | 0.5 |
| Methyl p-oxybenzoate | 0.1 |
| Peppermint type A mixed fragrance | 0.5 |
| material (Example 3) | |
| Water | balance |

### Example 6

A chewing gum (stick gum) of peppermint type with the following formulation was produced in a usual manner.

| Mixing components | % by weight |
|---|---|
| Stick gum base | 24.0 |
| Starch syrup | 13.0 |
| Powder sugar | 62.0 |
| Peppermint type B mixed fragrance | 1.0 |
| material (Example 3) | |
| Pigment | adequate amount |

### Example 7

A candy of spearmint type with the following formulation was produced in a usual manner.

| Mixing components | % by weight |
|---|---|
| Granulated sugar | 44.5 |
| Starch syrup | 41.0 |
| Water | 14.0 |
| Spearmint type mixed fragrance | 0.5 |
| material (Example 3) | |
| Pigment | adequate amount |

### INDUSTRIAL APPLICABILITY

As detailed in the above, the antibacterial flavor and fragrance composition and halitosis-inhibition flavor and fragrance composition of the invention contains at least one fragrance material selected from safe food fragrance materials having a long-term use experience, and exhibit an antibacterial activity and an inhibitory activity of the production of volatile sulfides by saliva when added to an oral care composition. Furthermore, by combining these suitably, a flavor and fragrance composition exhibiting a stronger antibacterial effect against dental bacteria and a halitosis-inhibition effect can be provided. In addition, the oral care composition containing these flavor and fragrance compositions provides toothpaste, mouthwash, and foods excellent in a halitosis-inhibition effect.

While the invention has been described in detail with reference to specific embodiments thereof, it will be apparent to one skilled in the art that various changes and modifications can be made therein without departing from the spirit and scope thereof.

This application is based on Japanese patent application No. 2002-177134 filed on Jun. 18, 2002, the entire contents thereof being hereby incorporated by reference.

1. A method of reducing halitosis, the method comprising adding component (A) and component (B) to an oral care composition,
wherein the component (A) is an antibacterial flavor and fragrance composition for adding to the oral care composition to inhibit halitosis-causing bacteria growth, and comprises at least one substance selected from hexylaldehyde, caryophyllene alcohol, cinnamicaldehyde, dihydroeugenol, farnesol, dihydrofarnesol, hinokitiol, isoeugenol, γ-undecalactone, d-limonene, o-methoxycinnamicaldehyde, β-pinene, γ-terpinene, terpinolene, nutmeg oil, dill weed oil, pine needle oil, spearmint oil, sweety oil, and Mentha Herb oil, and
the component (B) is a halitosis-inhibition flavor and fragrance composition for adding to the oral care composition to inhibit production of volatile sulfide, and comprises at least one substance selected from hexylaldehyde, allyl cyclohexylpropionate, allyl enanthate, α-amylcinnamicaldehyde, caryophyllene alcohol, l-carvone, cinnamicaldehyde, cinnamicaldehyde dimethyl acetal, citral, menthoxypropanediol, dihydroeugenol, ethyl salicylate, methyl salicylate, eugenol, farnesol, dihydrofarnesol, hinokitiol, isoeugenol, γ-decalactone, γ-undecalactone, d-limonene, linalyl acetate, menthol, o-methoxycinnamicaldehyde, methyl-isoeugenol, β-pinene, γ-terpinene, terpinolene, thymol, nutmeg oil, pine needle oil, spearmint oil, sweety oil, peppermint oil, eucalyptus oil, and Mentha Herb oil.

2. The method according to claim 1, wherein the at least one substance in the component (A) is different from the at least one substance in the component (B).

3. The method according to claim 1, wherein the component (A) is capable of reducing a bacterium to 1/10 or less in concentration.

4. The method according to claim 1, wherein the component (B) is capable of inhibiting production of volatile sulfide under a condition of 37° C. for three hours.
